(19) United States
(12) Patent Application Publication
(10) Pub. No.: US 2026/0183209 A1
(43) Pub. Date: Jul. 2, 2026

(54) USE OF SANDALORE FOR IMPROVING THE STRUCTURE OF HAIR

(71) Applicant: WELLA GERMANY GMBH, Darmstadt (DE)

(72) Inventors: Ingo RIEMANN, Darmstadt (DE); Christian SPRINGOB, Darmstadt (DE); Massimo MORINI, Darmstadt (DE)

(21) Appl. No.: 18/867,885

(22) PCT Filed: May 9, 2023
(86) PCT No.: PCT/EP2023/062281

§ 371 Date: Nov. 21, 2024

(30) Foreign Application Priority Data
May 25, 2022 (EP) 22175442.7

(51) Int. Cl. A61K 8/34; A61K 8/36; A61K 8/49; A61Q 5/00; A61Q 5/12; A61Q 7/00

(52) U.S. Cl. CPC A61K 8/342; A61K 8/361; A61K 8/4953; A61Q 5/002; A61Q 5/12; A61Q 7/00; A61K 2800/10; A61K 2800/30

(57) ABSTRACT

Sandal pentanol (3-methyl-5-(2,2,3-trimethylcyclopent-3-en-1-yl) pentan-2-ol), or compositions containing sandal pentanol may be used for improving hair structure. The compositions optionally may further contain caffeine as an additional active ingredient, and/or one or more saturated or unsaturated C10-C18-fatty acids, or salts thereof as an additional active ingredient. Improving hair structure may comprise increasing the cross-section of the hair. Improving hair structure may comprise increasing the expression of keratin, in particural the expression of keratin 85. Improving hair structure may comprise improving the structure of hair affected by age-related hair thinning.

### FIELD OF THE INVENTION

The present invention relates to improving hair structure. In embodiments, the present invention relates to a cosmetic composition for improving hair structure. The composition contains sandal pentanol (3-methyl-5-(2,2,3-trimethylcyclopent-3-en-1-yl) pentan-2-ol) as an active ingredient. Optionally, the composition may contain caffeine as an additional active ingredient. Optionally, the composition may contain one or more saturated or unsaturated C10-C18-fatty acids, or salts thereof as an additional active ingredient. For example, the composition may contain sandal pentanol, caffeine and one or more saturated or unsaturated C10-C18-fatty acids, or salts thereof as active ingredients. In another aspect, the present invention relates to using sandal pentanol (3-methyl-5-(2,2,3-trimethylcyclopent-3-en-1-yl) pentan-2-ol) for improving hair structure optionally in combination with caffeine and/or one or more saturated or unsaturated C10-C18-fatty acids.

### BACKGROUND OF THE INVENTION

The growth of human hair occurs in a known manner according to a natural growth rhythm including a growth phase (anagen), a transition phase (catagen), a resting phase (telogen) and a release phase (exogen). A normal natural hair cycle begins when a hair bottom end is ejected or cast away in exogen phase or prior to beginning of a new anagen phase. During the anagen phase the cells in the root of the hair are dividing rapidly so that new hair is formed, with a typical growth rate for hair on the scalp of approximately 0.3 to 0.4 millimeters per day. Anagen phase lasts about 2-6 years. Following the anagen phase, the catagen phase denotes a short transition phase wherein hair growth is stopped and the cells that produce hair growth are cut off from blood supply. Catagen phase lasts approximately ten days. During telogen phase, which lasts about 100 days, the individual hairs are still retained in the follicles but are no longer actively growing. Finally, at exogen phase the individual hairs are ejected or released. Exogen phase lasts approximately 2-5 months. During normal hair growth, 80 to 100 hairs fall out from the scalp per day. This number is an approximate value, which can occur over a shorter time and also can be greater or smaller, without causing fear regarding natural hair growth.

Any loss of hair volume over the time is generally perceived as “thinning of hair”, but such a volume loss may be caused by different phenomena. One obvious reason for perceived thinner hair is hair loss, i.e. the number or areal density of hair follicles producing hair decreases. Another reason for perceived thinner hair are alterations in the growth cycle. For example, the percentage of hairs in anagen phase and the duration of the anagen phase may diminish, resulting primarily in shorter hairs but perceived as an overall loss of hair volume. Another reason for perceived thinner hair is an alteration of the diameter or cross-section of the hair, i.e. a thinning of the individual hairs.

Structure and size of human head hair or scalp hair vary depending on ethnic origin. Asian hair has a round, even shape and an average diameter from 80 to 120 micrometers. Caucasian hair are oval in shape, with 60 to 80 micrometers considered a typical average diameter for hair. African hair has a flattened oval shape, with average diameters from 50 to 70 micrometers considered typical. Average diameters also vary depending on age, with increasing diameters until the early 40s, and after that age the average diameter decreasing with an increasing rate.

A variety of “hair-thickening” products are commercially available. One type of such products aims at stopping or at least reducing hair loss, which has nothing to do with improving hair structure, for example by increasing the diameter or cross-section of hair. Another type of such products aims at improving hair style, in order to create an impression of more volume and full-hair look, again without any impact on the diameter or cross-section of hair. A third type of such “hair-thickening” products in fact aims increasing the diameter or cross-section of hair, essentially by applying a coating to the hair. Such coating can impart a thicker look to hair already grown out of the scalp, but does not increase the diameter of cross-section of the hair as it grows from the keratogenous bulb. These commercially available products also do not improve hair structure by increasing keratin expression, such as expression of keratin 85.

Therefore, a need exists for care products suitable for improving hair structure, in particular comprising increasing the diameter or cross-section of hair, or improving keratin expression. Further, there is a need for care products suitable for improving the structure of hair affected by age-related hair thinning. In particular, there is a need for such products which can be administered topically.

EP0116439A2 discloses a hair tonic composition, comprising as an effective ingredient at least one of specified C10-C20 fatty acid, or a salt thereof. The hair tonic composition prevents the generation of dandruff in hair and itching in the scalp, and is said to accelerate the growth of hair.

US2002/0034485A1 discloses a hair tonic preventing or at least reducing hair loss, which hair tonic includes at least one saturated and unsaturated C10-C18 fatty acid, or a salt thereof, biotin and/or caffeine.

US2019/0216696A1 discloses the use of specified derivates of sandal pentanol for promoting hair growth and/or inhibiting or delaying hair loss in the human scalp.

WO2020/069522A1 discloses a cosmetic formulation comprising an effective amount of sandal pentanol or analogs thereof, and minoxidil or rosemary oil or emu oil or a combination thereof for the treatment and regrowth of mammalian hair loss.

### SUMMARY OF THE INVENTION

According to the present invention, it has now surprisingly been found that sandal pentanol is effective for improving hair structure. The present inventors found out that sandal pentanol is suitable for improving hair structure, wherein improving hair structure comprises increasing the cross-section of the hair. Further, the present inventors found out that sandal pentanol is suitable for improving hair structure, wherein improving hair structure comprises increasing the expression of keratin, in particural the expression of keratin 85. Further, the present inventors found out that sandal pentanol is suitable for improving the structure of hair affected by age-related hair thinning.

According to an aspect, subject matter of the present invention is a composition for improving hair structure, in particular the structure of of mammalian hair such as human hair. The composition comprises sandal pentanol. Optionally, the composition further comprises caffeine. Optionally, the composition further comprises one or more saturated and unsaturated C10-C18 fatty acids. Typically, the composition also comprises a solvent or solvent-water system.

The composition for improving hair structure optionally may further comprise one or more components that provide for a beneficial effect (denoted herein as “supportive components”), one or more oil components, one or more mineral components, and one or more cosmetically acceptable additives.

According to embodiments, the composition for improving hair structure is a hair tonic comprising a water-alcohol-based solvent system.

According to other embodiments, the composition for improving hair structure is a dry shampoo, in particular a dry shampoo to be applied in the form of an aerosol.

According to other embodiments, the composition for improving hair structure is a shampoo, for example a water-reduced shampoo.

According to embodiments, the composition is a ready-to-use scalp-and-hair treatment product for improving hair structure, which product is selected from hair tonic, hair lotion, hair conditioner, hair cream, hair shampoo, dry shampoo, hair rinse, hair liquid, hair oil, hair pomade, hair foam, hair spray.

According to another aspect, subject matter of the present invention is a method for improving hair structure, which method comprises applying sandal pentanol, or a composition according to the present disclosure to scalp (or a skin region comprising facial hair).

According to a further aspect, subject matter of the present invention is the use of sandal pentanol, or of a composition according to the present disclosure for improving hair structure.

The compositions, methods and uses of the present invention are suitable for improving hair structure. In an aspect, the compositions, methods and uses of the present invention are suitable for improving hair structure, wherein improving hair structure comprises increasing the cross-section of the hair. In another aspect, the compositions, methods and uses of the present invention are suitable for improving hair structure, wherein improving hair structure comprises increasing the expression of keratin, for example the expression of keratin 85. In yet another aspect, the compositions, methods and uses of the present invention are suitable for improving the structure of hair affected by age-related hair thinning.

The compositions, methods and uses of the present invention further are suitable for topical administration on the human scalp. The compositions, methods and uses of the present invention are suitable both for cosmetic and therapeutic use in improving hair structure.

### Definitions

In the specification that follows, the terms “sandal pentanol” and “Sandalore” are used interchangeably. Sandal pentanol is a synthetic odorant having a fragrance similar to sandalwood, and which consequently is used in perfumes, emollients and skin cleaning agents as a less expensive ingredient mimicking sandalwood scent. Sandal pentanol, as well as structurally similar analogs thereof such as the compound named brahmanol, are alcohols having a chemical structure quite distinct from the ingredients of natural sandalwood oil, which are extracted by steam distillation from matured sandalwood trees, and are well-known valued components for perfumes. The IUPAC denomination of sandal pentanol is 3-methyl-5-(2,2,3-trimethylcyclopent-3-en-1-yl) pentan-2-ol.

As used in the specification and the appended claims, the singular forms “a,” “an” and “the” include plural referents unless the context clearly dictates otherwise.

The term “may” in the context of this application means “is permitted to” or “is able to” and is a synonym for the term “can.” The term “may” as used herein does not mean possibility or chance.

The term “and/or” in the context of this application means one or the other or both. For example, an aqueous solution of A and/or B means an aqueous solution of A alone, an aqueous solution of B alone and an aqueous solution of a combination of A and B.

The term “about” is understood to mean±10 percent of the recited number, numbers or range of numbers. According to embodiments, the term “about” is understood to mean±2 percent of the recited number, numbers or range of numbers.

As used herein, the term “optionally” means that the corresponding step or feature may or may not be present. It includes both possibilities.

The terms “parts-by-weight” (PBW) and “parts-by-volume” (PBV) are used in their usual meaning. When used in a mixed context, for example an amount of a component indicated in PBW in admixture with a component indicated in PBV, the terms PBW and PBV as used herein denote the amount in grams of the component indicated in PBW relative to a volume in milliliters of the amount of the component indicated in PBV.

The term “composition” as used herein denotes a mixture or blend comprising at least two components. Typical compositions comprise at least one functional component (such as, for example, an active ingredient such as sandal pentanol) and at least one solvent component (such as water or an alcohol). A composition may be present in any suitable physical form, including for example liquids of varying viscosities, gels, foams, creams, and the like. The term “composition” may denote a ready-to-use product unless the context dictates otherwise.

“Ready-to-use scalp-and-hair treatment product” denotes the form in which the composition is actually applied to human scalp, or a skin region comprising facial hair.

The term “essentially free” of a particular component denotes an amount of less than 1.0% by weight of the respective component in an entity such as a composition, based on the total weight of the entity. In particular, the term “essentially free” denotes an amount of less than 0.5% by weight, for example less than 0.1% by weight, such as less than 0.01% by weight of the respective component in the entity.

The term “free” of a particular component denotes an amount below the detection limit, assuming that detectability is on a ppm (parts per million) basis (by weight). In particular, the term “free” of a particular component denotes an amount below 50 ppm by weight, for example less than 10 ppm by weight, such as less than 5 ppm by weight of the respective component in the entity.

The term “consisting essentially of” denotes the absence of additional, not specifically recited components in an entity such as a composition in an amount of more than 1.0% by weight, based on the total weight of the entity. In particular, the term “consisting essentially of” denotes the absence of additional, not specifically recited components in an amount of more than 0.5% by weight, for example more than 0.1% by weight, such as more than 0.01% by weight in an entity. It goes without saying that any additional, not specifically recited component is compatible with the specifically recited components and does not interfere with the function thereof.

The term “consisting of” denotes the absence of additional, not specifically recited components in an entity such as a composition in an amount below the detection limit, assuming that detectability is on a ppm (parts per million) basis (by weight). In particular, the term “consisting of” denotes the absence of additional, not specifically recited components in an amount of more than 50 ppm by weight, for example more than 10 ppm by weight, such as more than 10 ppm by weight in an entity. It goes without saying that any additional, not specifically recited component is compatible with the specifically recited components and does not interfere with the function thereof.

The term “hair” as used herein denotes generally mammalian hair, but in particular human hair. More specifically, for the purposes of the present invention the term “hair” denotes human “head hair” or “scalp hair”, and human “facial hair”. The term “facial hair” encompasses beard hair, eyebrows and eyelashes.

The term “improved hair structure” denotes an improvement in one or more structural phenomena of hair. For example, “improved hair structure” encompasses an improved/increased “diameter” or “cross-section” of the hair “as grown”. In another example, “improved hair structure” encompasses improved/increased keratin expression, in particular improved/increased expression of KRT85. The term “keratin 85” or “KRT85” denotes human keratin 85.

The term “age-related hair-thinning” denotes a perceived loss of the volume of human hair caused by a decrease of the diameter or cross-section of the hair, which decrease occurs in an age-dependent fashion with an onset age typically in the early 40's. Scientific studies have demonstrated that the age-dependent decrease of the diameter or cross-section of hair correlates with a decreased expression of keratins, including a decreased expression of KRT85.

The term “KRT85 Expression” denotes the expression of human keratin 85. Just beyond the proliferative zone with stem cells (many studies refer to as the ‘germative matrix’ or Zone A) KRT85 occurs as one of the first keratins expressed in the cortical cells forming short filaments (paired with KRT35 or KRT31) in the cytoplasm in the ‘precortical hair matrix’. KRT85 promotes lateral association of short intermediate filaments (IFs) into bundles. These initial bundles are the first signs of keratin ultrastructure and are normally found in the cortex about level with the top of the dermal papilla. These intermediate filaments are always found as tightly aligned bundles. As cortex cells move into Zone B final fibre diameter and shape is determined as the Henle's layer hardens (Inner Root Sheath). In Zone C (start of the keratogenous region) the final shape and position is maintained. This suggests that KRT85 expression correlates directly with hair shaft quality, with an increased KRT85 expression resulting in an increased cross-section of the hair.

### DETAILED DESCRIPTION OF THE INVENTION

The inventors of the present invention surprisingly found that sandal pentanol is effective for improving hair structure. The sandal pentanol optionally is used in combination with caffeine, and/or in combination with one or more C10-C18 fatty acid(s). The inventors of the present invention surprisingly found that sandal pentanol and caffeine and/or one or more C10-C18 fatty acid(s) are effective in a synergistic manner for improving hair structure.

### 1 Sandal Pentanol

Sandal pentanol is the active ingredient used according to the present disclosure for improving hair structure. Sandal pentanol is a synthetic odorant used as an ingredient mimicking sandalwood scent. Sandal pentanol is an alcohol having a chemical structure quite distinct from the ingredients of natural sandalwood oil which usually are extracted by steam distillation of wood from matured sandalwood trees. The IUPAC denomination of sandal pentanol is 3-methyl-5-(2,2,3-trimethylcyclopent-3-en-1-yl) pentan-2-ol. Sandal pentanol has been shown to have a stimulatory effect on the olfactory receptor OR2AT4, which is expressed in the epithelium of human hair follicles.

Analogs or derivatives, respectively, of sandal pentanol are believed to have a similar effect on improving hair structure, and may be used in the present disclosure alternatively or additionally to sandal pentanol. Sandal pentanol analogs suitable as active ingredients include:
(4Z)-3-methyl-5-(2,2,3-trimethylcyclopent-3-en-1-yl) pent-4-en-2-ol (sandal pentenol)
1-methyl-2-((1,2.2-trimethylbicyclo(3.1.0) hex-3-yl)methyl)-cyclopropane-methanol (sandal cyclopropane)
(E)-3,3-dimethyl-5-(2,2,3-trimethylcyclopent-3-en-1-yl) pent-4-en-2-ol (santol pentenol)
2-methyl-4-(2,2,3-trimethyl-3-cyclopenten-1-yl) butanol (sandal cyclopentane)
(E)-2-ethyl-4-(2,2,3-trimethylcyclopent-3-en-1-yl) but-2-en-1-ol (sandalrome)
(E)-2-methyl-4-(2,2,3-trimethyl-3-cyclopenten-1-yl)-2-buten-1-ol (sandal butenol)

According to embodiments, compositions according to the present disclosure may comprise one or more of the above sandal pentanol analogs in addition to the active ingredient sandal pentanol. If present, sandal pentanol plus the one or more sandal pentanol analogs are present in total amounts as indicated below for sandal pentanol alone.

According to other embodiments, compositions according to the present disclosure may comprise sandal pentanol as an active ingredient, and being essentially free of, or free of the above sandal pentanol analogs.

According to further, less preferred embodiments, compositions according to the present disclosure may comprise one or more of the above sandal pentanol analogs substituting for the active ingredient sandal pentanol.

The amount of sandal pentanol in the compositions according to the present disclosure is not particularly critical. Typically, the sandal pentanol will be present in an amount of from 0.010 to 10.0 percent-by-weight, based on the weight of the composition. If the amount is below 0.010 percent-by-weight, the effect sought by the present disclosure may be difficult to achieve. Amounts above 10.0 percent-by-weight may not further enhance the effect, but contribute to higher costs of the material resources for the compositions according to the present disclosure. According to embodiments, the sandal pentanol may be present in an amount of 0.050 to 5.0 percent-by-weight, based on the weight of the composition. According to more specific embodiments, the sandal pentanol may be present in an amount of 0.10 to 2.0 percent-by-weight, based on the weight of the composition. According to even more specific embodiments, the sandal pentanol may be present in an amount of 0.50 to 1.50 percent-by-weight, based on the weight of the composition.

If analogs of sandanol pentanol are present, either replacing at least in part the sandal pentanol, or in addition to sandal pentanol, the total amount of sandal pentanol plus one or more of sandal pentanol analogs (as indicated above) is within the ranges indicated above for sandal pentanol exclusively.

### 2 Optional Further Active Ingredients

Compositions according to the present disclosure comprise sandal pentanol, also denoted as Sandalore, and further may comprise as additional active ingredients one or both of:
caffeine, and
one or more saturated and/or unsaturated C10-C18 fatty acids.

Caffeine is an optional active ingredient used according to the present disclosure for improving hair structure. Caffeine is a plant alkaloid also known under the names theine, methyltheobromine and guaranine. The IUPAC denomination is 1,3,7-trimethylxanthine. Caffeine is a well-known stimulant which is present for example in coffee, black tea, but also in cocoa solids and products containing same (in particular dark chocolate), or cola nuts. Caffeine has a vasodilating effect, thereby increasing blood and nutrients supply to the hair roots. Studies have shown that caffeine enhances the hair shaft elongation, prolonges anagen duration (increasing the hair prowth phase) and stimulates the proliferation of hair matrix keratinocytes (increasing keratin production).

The amount of caffeine in the compositions according to the present disclosure is not particularly critical. If present, the caffeine typically will be present in an amount of from 0.010 to 10.0 percent-by-weight, based on the weight of the composition. If the amount is below 0.010 percent-by-weight, the effect sought by the present disclosure may be difficult to achieve. Amounts above 10.0 percent-by-weight may not further enhance the effect, but contribute to higher costs of the material resources for the compositions according to the present disclosure. According to embodiments, the caffeine may be present in an amount of 0.050 to 5.0 percent-by-weight, based on the weight of the composition. According to more specific embodiments, the caffeine may be present in an amount of 0.10 to 2.0 percent-by-weight, based on the weight of the composition. According to even more specific embodiments, the caffeine may be present in an amount of 0.50 to 1.5 percent-by-weight, based on the weight of the composition.

Another optional active ingredient used according to the present disclosure for improving hair structure are C10-C18 fatty acids. The fatty acid(s) optionally used for improving hair structure is/are one or more linear and/or branched, saturated and/or unsaturated C10-C18 fatty acids. Typically, the one or more C10-C18 fatty acids will be linear fatty acids. According to embodiments, the C10-C18 fatty acid(s) may be selected from capric acid (C10:0), lauric acid (C12:0), myristic acid (C14:0), isomyristic acid (C14:0), palmitic acid (C16:0), palmitoleic acid (C16:1), stearic acid (C18:0), oleic acid (C18:1), linoleic acid (C18:2), linolenic acid (C18:3), parinaric acid (C18:4), petroselinic acid (C18:1), or combinations thereof.

According to embodiments, the C10-C18 fatty acid(s) may comprise lauric acid (C12:0), myristic acid (C14:0), or a combination thereof. According to specific embodiments, the C10-C18 fatty acid(s) may be selected from lauric acid (C12:0), myristic acid (C14:0), or a combination thereof. For example, the fatty acid component of the active ingredient combination may be lauric acid (C12:0).

According to embodiments, the fatty acid component may comprise one or more unsaturated fatty acids. According to embodiments, the C10-C18 fatty acid(s) may comprise palmitoleic acid (C16:1), oleic acid (C18:1), linoleic acid (C18:2), linolenic acid (C18:3), parinaric acid (C18:4), petroselinic acid (C18:1), or a combination thereof. For example, the fatty acid component may comprise linoleic acid (C18:2).

According to specific embodiments, the fatty acid component may be selected from palmitoleic acid (C16:1), oleic acid (C18:1), linoleic acid (C18:2), linolenic acid (C18:3), parinaric acid (C18:4), petroselinic acid (C18:1), or a combination thereof. For example, the fatty acid component may be selected from palmitoleic acid (C16:1) and/or linoleic acid (C18:2).

If the fatty acid component comprises one or more unsaturated fatty acids, the respective compositions preferably comprise an antioxidant in order to reduce autoxidation of the double bonds.

According to embodiments, compositions according to the present disclosure may be essentially free, or free of unsaturated C10-C18 fatty acids. According to specific embodiments, compositions according to the present disclosure may be essentially free, or free of unsaturated fatty acids per se.

If present, the C10-C18 fatty acid(s) may be present in the form of free acid, or in the form of a cosmetically acceptable salt, for example in the form of an alkali metal salt, alkaline earth metal salt, ammonium salt or combination thereof. For example, if present the C10-C18 fatty acid(s) may be present in the form of the free acid(s).

The amount of C10-C18 fatty acid(s) in the compositions according to the present disclosure is not particularly critical. If present, the C10-C18 fatty acid(s) typically will be present in an amount of from 0.010 to 10.0 percent-by-weight, based on the weight of the composition. If the amount is below 0.010 percent-by-weight, the effect sought by the present disclosure may be difficult to achieve. Amounts above 10.0 percent-by-weight may not further enhance the effect, but contribute to higher costs of the material resources for the compositions according to the present disclosure. High amounts of C10-C18 fatty acid(s) further may require higher amounts of surfactants, and may result in a greasy appearance or hair feel. According to embodiments, the C10-C18 fatty acid(s) may be present in an amount of 0.050 to 5.0 percent-by-weight, based on the weight of the composition. According to more specific embodiments, the C10-C18 fatty acids may be present in an amount of 0.10 to 2.0 percent-by-weight, based on the weight of the composition. According to even more specific embodiments, the C10-C18 fatty acids may be present in an amount of 0.50 to 1.5 percent-by-weight, based on the weight of the composition.

### 3 Relative Amounts of Active Ingredients in Combination

Typically, sandal pentanol will be present in an amount of from 0.010 to 10.0 percent-by-weight, based on the weight of the composition. Caffeine, if present, typically will be present in an amount of from 0.010 to 10.0 percent-by-weight, based on the weight of the composition. One or more C10-C18 fatty acid(s), if present, typically will be present in an amount of from 0.010 to 2.0 percent-by-weight, based on the weight of the composition. Conveniently, the sandal pentanol will be present in an amount of from 0.10 to 5.0 percent-by-weight, the caffeine will be present in an amount of from 0.10 to 5.0 percent-by-weight, and the one or more C10-C18 fatty acid(s) will be present in an amount of from 0.050 to 3.0 percent-by-weight, each based on the weight of the composition.

According to embodiments, the sandal pentanol will be present in an amount of from 0.50 to 2.0 percent-by-weight, the caffeine will be present in an amount of from 0.50 to 2.0 percent-by-weight, and the one or more C10-C18 fatty acid(s) will be present in an amount of from 0.10 to 2.0 percent-by-weight, each based on the weight of the composition. According to more specific embodiments, the sandal pentanol will be present in an amount of from 0.80 to 1.50 percent-by-weight, the caffeine will be present in an amount of from 0.80 to 1.50 percent-by-weight, and the one or more C10-C18 fatty acid(s) will be present in an amount of from 0.10 to 1.0 percent-by-weight, each based on the weight of the composition.

According to embodiments encompassing the combination of sandal pentanol, caffeine and/or one or more C10-C18 fatty acid(s), the weight ratio of sandal pentanol to caffeine may be from 1:2 to 2:1, more specifically from 1:1.5 to 1.5:1, and in particular from 1:1.2 to 1.2:1. In such embodiments, the weight ratio of sandal pentanol to the one or more C10-C18 fatty acid(s) may be from 10:1 to 1:1, more specifically from 8:1 to 1.5:1, and in particular from 5:1 to 2:1. Further, the weight ratio of caffeine to the one or more C10-C18 fatty acid(s) analogously may be from 10:1 to 1:1, more specifically from 8:1 to 1.5:1, and in particular from 5:1 to 2:1. Given that the molecular weights of sandal pentanol, caffeine and C10-C18 fatty acids are rather similar, molecular ratios will be similar to the weight ratios.

### 4 Optional Added-Benefit Components

Compositions according to the present disclosure may optionally comprise additional components which enhance, supplement or add to the benefit obtained by sandal pentanol alone or an active ingredient combination. Such additional optional components will be referred to in the following as supportive component, oil component and mineral component. The supportive component covers a broad spectrum of substances having diversified influences on physiologic or metabolic processes. The oil component covers natural essential oils and modified oils. Modified oils include in particular hydrogenated oils and oil constituents. The mineral component covers minerals in water-soluble or alcohol-soluble form.

According to embodiments, compositions according to the present disclosure further may comprise a supportive component. Examples of such supportive components include in particular vasodilatators, blood circulation stimulators, antioxidants, vitamins, provitamins. If present, the amount of the supportive component or combinations thereof will be in the range of from 0.0010 to 5.0, in particular from 0.010 to 3.0 percent-by-weight, based on the weight of the composition.

The supportive component may comprise, for example, one or more of tocopherols, tocotrienols, tocopherol esters, nicotinic acid, nicotinic amides, nicotinic esters, vasodilatory plant extracts, camphor, royal jelly, biotin, vitamin B3, vitamin B12, vitamin C, vitamin D2, vitamin D3, vitamin E, vitamin K1, vitamin K2, panthenol (provitamin B5), omega-3 fatty acids, omega-6 fatty acids, folates, cannabidiol, amino acids, or combinations thereof.

According to embodiments, the supportive component includes a vasodilator selected from tocopherols (alpha-, beta-, gamma- or delta-tocopherol), in particular alpha-tocopherol, tocopherol esters (including, for example, tocopherol acetate, tocopheryl succinate, tocopherylnicotinate, tocopherylpoly(oxyethylene)-succinate), nicotinic acid, nicotinic amides, nicotinic esters, camphor; an antioxidant such as vitamin C or polyphenol(s); an inhibitor of testosterone-5-alpha-reductase such as the omega-6 fatty acid arachidonic acid; a substance providing a benefit to hair health such as nicotinic amide (niacinamide); or a combination thereof. According to embodiments, the supportive component includes tocopherol or tocopheryl acetate (vitamin E acetate), and/or niacinamide. Suitable vasodilatory plant extracts include burned or distilled nettle extract (Urtica Diocia), roast chestnut extract (Aesculus Hippocastanum), Arnica extract (Arnica Montana), or hemp extract.

The oil component may comprise, for example, peppermint oil, rosemary essential oil, lavender essential oil, tea tree oil, ylang-ylang, thyme oil, cedarwood oil, clary sage essential oil, bergamot oil, jojoba oil, sunflower oil, hydrogenated ethylhexyl olivate, or a combination thereof. If present, the oil component will be present in the compositions of the present disclosure in an amount of from 0.0010 to 1.0, in particular from 0.010 to 0.50 percent-by-weight, based on the weight of the composition.

According to embodiments, the oil component includes peppermint oil and/or hydrogenated ethylhexyl olivate.

The mineral component includes metal ions having a beneficial effect on hair and/or scalp health. The mineral component may comprise, for example, iron, zinc, or a combination thereof. If present, the mineral component will be present in the compositions of the present disclosure in 0.0050-0.50 molar concentration, for example in 0.010-0.25 molar concentration.

Compositions according to the present disclosure may comprise a supportive component, an oil component or a mineral component alone or in any combination.

### 5 the Solvent or Carrier System, and pH Range

The solvent system used in the compositions of the present disclosure is water-based, alcohol-based or water-alcohol-based. Typically, the solvent comprises water and one or more monovalent and/or polyvalent C1-C4 alcohols. According to embodiments, the solvent comprises water and an alcohol selected from ethanol, n-propanol, isopropanol, glycerol, ethylene glycol, propylene glycol or a combination thereof.

The solvent further may comprise one or more solubilisation enhancers. The solubilisation enhancers obviously are intended to solubilize compounds which are not or only partially soluble in the solvent at the concentration used in the compositions. Solubilisation enhancers generally include surfactants. Surfactants include anionic, cationic, amphoteric and non-ionic surfactants.

The anionic surfactants may be selected from the group consisting of salts (such as alkaline salts, for example, sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, alpha-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates; N-acyltaurates; and mixtures thereof. The alkyl or acyl radical of all of these various compounds, for example, comprises from 8 to 24 carbon atoms, and the aryl radical, for example, is chosen from phenyl and benzyl groups. Weakly anionic surfactants can also be used, such as alkyl-D-galactosiduronic acids and their salts, as well as polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, for example, those comprising from 2 to 50 ethylene oxide groups, and mixtures thereof. Anionic derivatives of polysaccharides, for example carboxyalkyl ether of alkyl polyglucosides, can be also used.

Suitable anionic surfactant(s) may comprise at least one anionic functional groups at their head selected from sulfate, sulfonate, phosphate and carboxylates. In order to avoid any overlap with an active ingredient combination of the present disclosure, anionic surfactant(s) for use in the compositions according to the present disclosure exclude fatty acids or salts thereof.

Suitable alkyl sulfates include ammonium lauryl sulfate, sodium lauryl sulfate (sodium dodecyl sulfate, SLS, or SDS), and alkyl-ether sulfates, such as sodium laureth sulfate (sodium lauryl ether sulfate or SLES), and sodium myreth sulfate.

Further suitable anionic surfactants may include docusate (dioctyl sodium sulfosuccinate), alkyl-aryl ether phosphate, alkyl ether phosphate, sodium lauroyl sarcosinate, ammonium laureth sulfate, disodium lauryl sulfosuccinate, and sodium lauryl sulphoacetate.

Preferred anionic surfactants may be selected from the group consisting of sodium laurylethersulfate, sodium laurethethersulfate, sodium dodecyl sulfate, ammonium laurethethersulfat, ammonium dodecyl sulfate, alkylbenzenesulfonate, and combinations thereof.

The surfactant may be a non-ionic surfactant. The non-ionic surfactant may be selected from the group consisting of lanolin alcohol, and polyoxyethylene ethers of fatty alcohols, and mixtures thereof. The non-ionic surfactant may be preferably ceteareth-n, wherein n is from 2 to 100, or from 10 to 30. Suitable nonionic surfactants are compounds that are well known (see, for example, in this respect “Handbook of Surfactants” by M. R. Porter, published by Blackie & Son (Glasgow and London), 1991, pp. 116-178).

Preferred surfactants encompass non-ionic surfactants, in particular polyoxyethylated fatty alcohols or oils. Particularly preferred non-ionic surfactants include polyoxyethylated Castor Oil and hydrogenated polyoxylated Castor Oil. Specific examples of non-ionic surfactants that may be used in the compositions of the present disclosure are PEG-35 Castor Oil, PEG-40 Hydrogenated Castor Oil, and combinations thereof.

Compositions of the present disclosure can have a pH within the range of from 3.0 to 8.0. According to embodiments, the pH may be within the range of from 4.0 to 6.5, for example within the range of from 4.5 to 6.0.

### 6 Optional Cosmetically Acceptable Additives

Compositions according to the present disclosure optionally may comprise cosmetically acceptable additives, which may contribute to achieve or enhance the properties or effects sought. According to embodiments, the cosmetically acceptable additives may be selected from perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents. The cosmetically acceptable additives may be used in any combinations, as desired or required.

Suitable cosmetically acceptable additives not specifically described below are listed in the International Cosmetics Ingredient Dictionary and Handbook, (8th ed.; The Cosmetics, Toiletry, and Fragrance Association). Particularly, vol. 2, sections 3 (Chemical Classes) and 4 (Functions), which are useful in identifying specific additives to achieve a particular purpose or multipurpose. A few of these additives are discussed below, whose disclosure is of course non-exhaustive.

Suitable amount ranges for perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents are indicated for additives discussed below. These cosmetically acceptable additives may be present in a total amount of up to 20.0, in particular up to 15.0 percent-by-weight, based on the weight of the composition. Typically, the total amount of these cosmetically acceptable additives may be within a range of from 0.010 to 10.0, conveniently within a range of from 0.010 to 7.0 percent-by-weight, based on the weight of the composition. For example, the total amount of these cosmetically acceptable additives may be within a range of from 0.010 to 5.0, conveniently within a range of from 0.010 to 3.0 percent-by-weight, based on the weight of the composition.

### 6.1 Perfume or Fragrance

Compositions according to the present disclosure may comprise a perfume or fragrance. For example, the compositions may comprise from 0.0010 to 1.50, in particular 0.010-0.50 percent-by-weight perfume, based on the weight of the composition. Perfume can provide an enhanced user experience by making the composition smell pleasant and/or invoke emotions, such as relaxing or exciting smells.

Alternatively, compositions according to the present disclosure may be substantially free of perfume and/or fragrance. Some consumers prefer perfume-free compositions.

The perfume may be an animal fragrance or a plant fragrance. The animal fragrance may be selected from the group consisting of musk oil, civet, castoreum, ambergris, and mixtures thereof.

The plant fragrance may be selected from the group consisting of nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, and mixtures thereof.

The perfume may comprise one or more scents selected from the group consisting of acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronnellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitrolnellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-α-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox, and mixtures thereof.

### 6.2 Opacifiers or Turbidity-Inducing Agents

Compositions in accordance with the present disclosure may comprise an opacifier, which sometimes is denoted as well by the term “turbidity-inducing agent”. For example, the compositions may comprise from 0.10 to 5.0, in particular 0.50-2.0 percent-by-weight opacifier, based on the weight of the composition. Suitable opacifiers include mineral pigments such as, for example, titanium dioxide, alumina, calcium carbonate. Other suitable opacifiers include fatty alcohols and fatty acid esters such as, for example, cetearyl alcohol, cetyl alcohol, ethylene glycol distearate, glyceryl stearate, glyceryl oleate, stearyl stearate.

### 6.3 Preservatives

Compositions in accordance with the present disclosure may comprise a preservative or mixture of preservatives. The compositions may comprise the preservative(s) in an amount of up to 1.0, for example from 0.0010 to 1.0, in particular from 0.010 to 0.50 percent-by-weight, based on the weight of the composition. Cosmetically acceptable preservatives include organic acids such as para-hydroxybenzoic acid; organic acid salts such as sodium benzoate; compounds such as benzyl alcohol, phenoxyethanol, 1,3-bis(hydroxymethyl)-5,5-dimethylimidazolidine-2,4-dione; and mixtures thereof.

### 6.4 pH Modifiers

Compositions in accordance with the present disclosure may comprise a pH modifier and/or buffering agent, in a sufficient amount to effectively adjust the pH of the composition. For example, the compositions may comprise up to 1.0, for example from 0.000010 to 1.0, in particular 0.0010-0.50 percent-by-weight pH modifiers, based on the weight of the composition. Suitable pH modifiers and/or buffering agents for use herein include, but are not limited to: ammonia, alkanolamines such as monoethanolamine, diethanolamine, triethanolamine, monopropanolamine, dipropanolamine, tripropanolamine, tripropanolamine, 2-amino-2-methyl-1-propanol, and 2-amino-2-hydroxymethyl-1,3,-propandiol and guanidium salts, alkali metal and ammonium hydroxides and carbonates, such as sodium hydroxide, sodium silicate, sodium meta silicate and ammonium carbonate, and acids such as organic and inorganic acids, for example ascorbic acid, citric acid, acetic acid or tartaric acid, phosphoric acid, hydrochloric acid, and mixtures thereof.

Preferred alkaline pH modifiers include ammonia, ethanolamines, and alkali metal hydroxides, in particular sodium hydroxide. Preferred acidic pH modifiers include hydrochloric acid, ascorbic acid, or citric acid.

### 6.5 Chelators

Compositions according to the present disclosure may further comprise one or more chelators (also known as “chelating agent”, “sequestering agent”, or “sequestrant”) in an amount sufficient to reduce the amount of metals available to interact with formulation components. Chelators are well known in the art and a non-exhaustive list thereof can be found in AE Martell & RM Smith, Critical Stability Constants, Vol. 1, Plenum Press, New York & London (1974) and AE Martell & RD Hancock, Metal Complexes in Aqueous Solution, Plenum Press, New York & London (1996). Typically, the compositions may comprise one or more chelators in a total amount of from 0.0010 to 1.0, in particular from 0.010 to 0.50 percent-by-weight, based on the weight of the composition.

The one or more chelators may be selected from the group consisting of carboxylic acids (such as aminocarboxylic acids), phosphonic acids (such as aminophosphonic acids), polyphosphoric acids (such as linear polyphosphoric acids), their salts thereof, and mixtures thereof.

By “salts thereof”, it is meant—in the context of chelators-all salts comprising the same functional structure as the chelators they are referring to and including alkali metal salts, alkaline earth salts, ammonium salts, substituted ammonium salts, and mixtures thereof; alternatively sodium salts, potassium salts, calcium salts, magnesium salts, ammonium salts, and mixtures thereof; alternatively monoethanolammonium salts, diethanolammonium salts, triethanolammonium salts, and mixtures thereof.

The one or more chelators may be one or more aminocarboxylic acid chelators comprising one or more carboxylic acid moieties (—COOH) and one or more nitrogen atoms. The one or more aminocarboxylic acid chelators may be selected from the group consisting of diethylenetriamine pentaacetic acid (DTPA), ethylenediamine disuccinic acid (EDDS), ethylenediamine-N,N′-diglutaric acid (EDDG), 2-hydroxypropylenediamine-N-N′-disuccinic acid (HPDDS), glycinamide-N,N′-disuccinic acid (GADS), ethylenediamine-N-N′-diglutaric acid (EDDG), 2-hydroxypropylenediamine-N-N′-disuccinic acid (HPDDS), ethylenediaminetetraacetic acid (EDTA), ethylenedicysteic acid (EDC), ethylenediamine-N-N′-bis(ortho-hydroxyphenyl acetic acid) (EDDHA), diaminoalkyldi (sulfosuccinic acids) (DDS), N,N′-bis(2-hydroxybenzyl)ethylenediamine-N,N′-diacetic acid (HBED), their salts thereof, and mixtures thereof. Conveniently, a chelator used in compositions according to the present disclosure, if any, may be EDTA.

### 6.6 Viscosity Modifiers or Rheology Modifiers

Compositions in accordance with the present disclosure may comprise viscosity modifiers, referred to in the art as well as “thickening agents”. If present, such viscosity modifiers typical will be used in the compositions in an amount of from 0.010 to 3.0, in particular from 0.50 to 1.0 percent-by-weight, based on the weight of the composition. Suitable cosmetically acceptable viscosity modifiers include non-ionic polymers, for example polysaccharides such as cellulose, cellulose gum or xanthan gum, and non-ionic viscosity modifiers based on polyether-1 such as the commercially available viscosity modifiers Pure Thix 1442.

### 6.7 Further Cosmetically Acceptable Additives

Compositions in accordance with the present disclosure may further comprise care materials, such as hair-care providing or skin-care-providing plant or vegetable extracts, cationic polymers or cationic surfactants. Typical amounts of care materials in the compositions according to the present disclosure will be in the range of from 0.010 to 5.0, in particular 0.050-2.0 percent-by-weight, based on the weight of the composition.

Additional cosmetically acceptable additives, which may be present in the compositions according to the present disclosure include:
physiologically compatible silicone derivative compounds, such as volatile or non-volatile silicone or high molecular weight siloxane polymers, such as for example Quaternium-80, typically in an amount of from 0.05 to 20 percent-by-weight, in particular from 0.10 to 5.0 percent-by-weight, based on the weight of the composition
light protecting agents, such as for example octylmethoxy cinnamate
anti-flaking agents, typically in an amount of about 0.01 to 2 percent-by-weight
hair luster-imparting agents, such as for example phenyltrimethicone,
combability improving agents, such as for example cationic surfactants such as Behentrimonium Chloride, or cationic polymers such as Polyquaternium-10
defatting agents
conditioning agents
moisturizing agents, such as for example sodium lactate, sodium PCA, glycine, fructose, urea, niacinamide, inositol
cooling agents, such as for example menthol, eucalptol, geraniol, linalool, isopulegol, cubedol, p-mentane-3,8-diols, hydroxy-citronellal as well as esters and derivatives thereof; ketones such as menthone and carvone; carboxamides, or evaporative cooling agents

The cosmetically acceptable additives may be used in the compositions according to the present disclosure in any combinations, as desired or required.

### 7 Substances Potentially Excluded

Compositions according to the present disclosure may be essentially free of, or be free of certain substances or classes of chemical compounds.

According to embodiments, compositions according to the present disclosure may be essentially free of biotin. According to embodiments, compositions according to the present disclosure may be free of biotin. According to particular embodiments, the concentration of biotin may be less than 5 ppm by weight.

According to embodiments, compositions according to the present disclosure may be essentially free of quaternary ammonium compounds. According to embodiments, compositions according to the present disclosure may be free of quaternary ammonium compounds. According to particular embodiments, the concentration of quaternary ammonium compounds may be less than 5 ppm by weight.

According to embodiments, compositions according to the present disclosure may be essentially free of silicones. According to embodiments, compositions according to the present disclosure may be free of silicones. According to particular embodiments, the concentration of silicones may be less than 5 ppm by weight.

According to embodiments, compositions according to the present disclosure may be essentially free of quaternary ammonium compounds and silicones. According to embodiments, compositions according to the present disclosure may be free of quaternary ammonium compounds and silicones. According to particular embodiments, the concentration of quaternary ammonium compounds and silicones may be less than 5 ppm by weight.

According to embodiments, the polymeric compounds present in compositions according to the present disclosure exclusively are film-forming polymers. In other words, according to embodiments, compositions according to the present disclosure may be essentially free of non-film-forming polymers. According to embodiments, compositions according to the present disclosure may be free of non-film-forming polymers. According to particular embodiments, the concentration of non-film-forming polymers may be less than 5 ppm by weight.

According to embodiments, compositions according to the present disclosure may be essentially free of or be free of non-film-forming polymers selected from acrylates/Steareth-20 methacrylate copolymer, carbomer, polyacrylamide, hydroxyethylcellulose, Xanthan Gum and polymers having similar chemistry.

According to embodiments, compositions according to the present disclosure may be essentially free of or be free of unsaturated fatty acids.

### 8 Specific Composition Embodiments

The following describes embodiments of compositions according to the present disclosure formulated as liquid formulation or aerosol.

### 8.1 Liquid Formulation Embodiments

According to embodiments, the compositions according to the present disclosure may be formulated as liquid formulations. The liquid formulation may have a conventional base for a cosmetic composition, for example an aqueous base, an alcoholic base or an aqueous-alcoholic base. The composition in the form of a liquid formulation may comprise (in addition to sandal pentanol or an active ingredient combination), for example, 70.0 to 99.5 percent-by-weight water-alcohol-based solvent. In particular, the composition in the form of a liquid formulation may comprise, for example, 90.0 to 99.0 percent-by-weight, for example 92.0 to 98.0 percent-by-weight, based on the weight of the composition, water-alcohol-based solvent.

The composition in the form of a liquid formulation may comprise, for example, water in an amount of at least 10 percent-by-weight, based on the weight of the composition, and alcohol in an amount of at least 20 percent-by-weight, based on the weight of the composition. The solvent may comprise, for example, lower univalent alcohols suitable for cosmetic purposes, such as ethanol, n-propanol and isopropanol, or multivalent alcohols, such as glycerol, ethylene glycol, propylene glycol, or a combination thereof. The composition in the form of a liquid formulation may further comprise solubilisation enhancers such as for example non-ionic surfactants. The pH of the composition in the form of a liquid formulation may be within the range of from 3 to 7, in particular within the range of from 4 to 6.5, for example within the range of from 4.5 to 6. Such composition in the form of a liquid formulation may be a hair tonic.

According to embodiments, the composition in the form of a liquid formulation may comprise a water-based solvent, and one or more surfactants in an amount of 5.0 to 25.0 percent-by-weight, based on the weight of the composition, wherein the surfactant(s) comprise(s) at least one anionic surfactant. Such composition in the form of a liquid formulation may be a shampoo.

According to embodiments, the composition in the form of a liquid formulation may comprise an alcohol-based or water-alcohol-based solvent, and one or more surfactants in an amount of 5.0 to 45.0 percent-by-weight, based on the weight of the composition, wherein the surfactant(s) comprise(s) at least one anionic surfactant. The solvent may comprise, for example, lower univalent alcohols suitable for cosmetic purposes, such as ethanol, n-propanol and isopropanol, or multivalent alcohols, such as glycerol, ethylene glycol, propylene glycol, or a combination thereof. Such composition in the form of a liquid formulation may be a shampoo.

### 8.2 Aerosol Formulation Embodiments

According to embodiments, compositions according to the present disclosure may be formulated as aerosol formulations. The compositions in the form of an aerosol formulation may comprise (in addition to sandal pentanol or an active ingredient combination), for example, 70.0 to 99.5 percent-by-weight, in particular 90.0 to 99.0 percent-by-weight, for example 92.0 to 98.0 percent-by-weight, based on the weight of the composition, of a blend comprising starch particles and an alcohol-based solvent.

The starch may be derived from any plant, for example potatoe, rize, corn, or tapioca. According to embodiments, the starch may be rice starch, tapioca starch, or a combination thereof. The starch particles may be hydrophobically modified starch particles, hydrophobically unmodified starch particles, or a combination thereof.

The alcohol-based solvent may comprise, for example, lower univalent alcohols suitable for cosmetic purposes, such as ethanol, n-propanol and isopropanol, or multivalent alcohols, such as glycerol, ethylene glycol, propylene glycol, or a combination thereof. According to particular embodiments, the alcohol(s) may be selected from ethanol and isopropanol.

The blend may further comprise a propellant, such as propane, isobutane, n-butane, cyclopropane, or a combination thereof. The propane may comprise halogenated hydrocarbone, which in view of environmental concerns is less preferred, however. The propellant is preferably miscible with the alcohol-based solvent.

The blend of the compositions in the form of an aerosol formulation may further comprise inorganic particles. Such inorganic particles act as a dispersing agent to suspend any dispersed solids or liquids within the composition. Including inorganic particles in the aerosol composition may separate the starch particles upon settling between uses, thereby preventing the creation of particle aggregates that are difficult to disrupt mechanically (by shaking). Suitable inorganic particles for the aerosol blends include silicas, clays, and mixtures thereof. For example, hydrophilic fumed silica particles having a total surface area of greater than 100 m2/g are suitable as inorganic particles for the aerosol blends.

According to embodiments, the blend may comprise, based on the weight of the blend, 5.0 to 12.0 percent-by-weight starch particles, 30.0 to 50.0 percent-by-weight alcohol-based solvent, 40.0 to 60.0 percent-by-weight propellant, and optionally 0.50 to 1.0 percent-by-weight inorganic particles. According to more specific embodiments, the blend may comprise, based on the weight of the blend, 7.0 to 10.0 percent-by-weight starch particles, 33.0 to 38.0 percent-by-weight alcohol-based solvent, 52.0 to 60.0 percent-by-weight propellant, and optionally 0.60 to 0.90 percent-by-weight inorganic particles.

According to embodiments, the composition in the form of an aerosol formulation may be a dry shampoo.

### 8.3 Scalp-and-Hair Treatment Product

According to embodiments, the compositions according to the present disclosure may be formulated as ready-to-use scalp-and-hair treatment products. For example, the compositions according to the present disclosure may be a ready-to-use scalp-and-hair treatment product selected from hair tonic, hair lotion, hair conditioner, hair cream, hair shampoo, dry shampoo, hair rinse, hair liquid, hair oil, hair pomade, hair foam, hair spray. The ready-to-use scalp-and-hair treatment products as well may be used for skin region comprising facial hair, or similar products may be formulated for skin regions comprising facial hair. When referring to “scalp” in the following, it is understood that such reference applies equally to part of the scalp region, or a skin region comprising facial hair, or part of such skin region.

According to preferred embodiments, after application the compositions according to the present disclosure are left on the scalp for a time sufficient to trigger the effect of sandal pentanol, or of an active ingredient combination according to the present disclosure. For example, the compositions may be left on scalp (or a skin region comprising facial hair) for at least 5 minutes, for example at least 15 minutes. The compositions may be left on scalp for at least 30 minutes, for example at least 60 minutes.

According to preferred embodiments, the ready-to-use scalp-and-hair treatment product is a leave-on product. Leave-on products are applied to hair and/or scalp (or a skin region comprising facial hair), and are not subsequently actively removed.

### 9 Methods and Uses

Sandal pentanol, or compositions according to the present disclosure may be employed according to a method wherein the sandal pentanol or the composition is applied to at least part of the scalp region, or to at least part of a skin region comprising facial hair. Conveniently, the sandal pentanol or the composition may be applied to at least part of the scalp region, or to at least part of a skin region comprising facial hair and subsequently or simultaneously be gently worked in.

Sandal pentanol, or compositions according to the present disclosure are applied in an amount sufficient to obtain the desired effect. In particular, the sandal pentanol or the composition may be applied applied to at least part of the scalp region, or to at least part of a skin region comprising facial hair in order to deliver an effective amount of sandal pentanol, or of an active ingredient combination. For example, sandal pentanol or compositions according to the present disclosure may be applied to at least part of the scalp region, or to at least part of a skin region comprising facial hair in one, or two, or even three or more applications per day in order to daily deliver a effective amount of sandal pentanol, or of an active ingredient combination. For example, sandal pentanol or compositions according to the present disclosure may be applied to at least part of the scalp region, or to at least part of a skin region comprising facial hair once or twice per day (24 hours).

Sandal pentanol, or compositions according to the present disclosure are left on at least part of the scalp region, or on at least part of a skin region comprising facial hair for sufficient time to obtain the desired effect. In particular, the sandal pentanol or the composition may be used as leave-on products, which after application to hair and/or at least part of the scalp region, and/or at least part of a skin region comprising facial hair are not- or at least not immediately-rinsed or washed out.

Further subject matter of the present disclosure is the use of sandal pentanol or of compositions according to the present disclosure for improving hair structure. For example, the present invention contemplates using sandal pentanol, or compositions according to the present disclosure, for improving hair structure, wherein improving hair structure comprises increasing the cross-section of the hair. Further, the present invention contemplates using sandal pentanol, or compositions according to the present disclosure, for improving hair structure, wherein improving hair structure comprises increasing the expression of keratin, in particural the expression of keratin 85. Further, the present invention contemplates using sandal pentanol, or compositions according to the present disclosure, for improving the structure of hair affected by age-related hair thinning.

### 9 Determination Methods

### 9.1 Cross-Section

The “cross-section” or “diameter” of hair is determined by the microscopic sectioned, newly formed hair shaft diameter in the keratogenous region (in a distance of around 200-350 μm from the ‘germative matrix’). In an ex vivo human scalp skin culture and treatment, 6 mm skin punches (hairy skin from a healthy donor) were made following the hair growth orientation and maintained in normoxic (37° C., 5% CO₂) condition. After 24 h resting (and then keeping) the culture in an Air-Liquid Interphase, two topical treatment were performed (day 1 and 3). Punches were embedded in an optimal cutting temperature compound after 5 days of ex vivo culture and cryosectioned (7 μm sections). The average value is calculated from 5 independent measurements for each hair follicle (HF).

### 9.2 Keratin 85 Expression

Expression of keratin 85 (KRT85) is set by fluorescence intensity in the precortical hair matrix with a fluorescence microscope at 20× magnification. Skin sections were co-stained o.n. at 4° C. with a guinea pig anti-KRT85 antibody (Progen, 1:200) followed by DAPI (IUPAC: 4′,6-Diamidino-2-phenylindol) counterstain to visualize the nuclei. The mean KRT85 expression value was calculated from all hair follicles with Image J software within each donor, and fold changes values versus vehicle group were determined.

### Examples

The following examples should illustrate the invention claimed below in greater detail, but the details of these examples should not be considered as limiting the claims appended hereinbelow.

### Example 1 Cross-Section

Example 1 demonstrates the increase of cross-section or hair shaft diameter by treatment with Sandalore alone, or a combination of Sandalore, caffeine and lauric acid, as compared to “vehicle”, i.e. negative control. In an ex vivo human scalp skin culture, punches (hairy skin from a human donors) were twice (day 1 and 3) treated topically, and analysed via crysectioning after 5 days. The average value for each hair follicle (HF) is calculated from 5 independent measurements, and the following table indicates the mean of all average values over the total number of hair follices (#HFs) measured.

| | hair shaft | |
|---|---|---|
| | diameter | |
| | [μm] | #HFs |
| Vehicle | 48.2 | n = 8 |
| Sandalore | 51.1 | n = 15 |
| Sandalore + caffeine + | 51.4 | n = 12 |
| lauric acid | | |

Sandalore alone increases the hair shaft diameter significantly, as compared to vehicle. The combination of Sandalore, caffeine and lauric acid increases the hair shaft diameter by 6.5% slightly succeeding the Sandalore only treatment.

### Example 2 Keratin 85 Expression

Example 2 demonstrates increased KRT85 expression of Sandalore via fluorescence intensity measurements in the ‘precortical hair matrix with a guinea pig anti-KRT85 antibody (Progen, 1:200) staining countered by DAPI. The mean KRT85 expression value expressed as fold changes values of Sandalore is significantly higher versus vehicle group.

| | change vs | |
|---|---|---|
| | vehicle | |
| | group | #HFs |
| Vehicle | 1 | n = 19 |
| Sandalore | 1.347 | n = 22 |
| Sandalore + caffeine + | 1.402 | n = 26 |
| lauric acid | | |

The change in KRT85 expression achieved by the combination of Sandalore, caffeine and lauric acid is higher as compared to Sandalore alone. In particular, the increase of KRT85 expression achieved by the combination of Sandalore, caffeine and lauric acid is plus 40% versus the vehicle group.

The following statements further illustrate the present invention.
1 Composition comprising sandal pentanol (3-methyl-5-(2,2,3-trimethylcyclopent-3-en-1-yl) pentan-2-ol), for improving hair structure.
2 The composition of statement 1, wherein improving hair structure comprises increasing the cross-section of the hair.
3 The composition of statement 1 or 2, wherein improving hair structure comprises increasing the expression of keratin.
4 The composition of statement 3, wherein improving hair structure comprises increasing the expression of keratin 85.
5 The composition of any of statements 1 to 4, for improving the structure of hair affected by age-related hair thinning.
6 The composition of any of the preceding statements, further comprising caffeine, one or more saturated and/or unsaturated C10-C18 fatty acids, or a combination thereof.
7 The composition of statement 6, wherein the C10-C18 fatty acid(s) is/are selected from capric acid (C10:0), lauric acid (C12:0), myristic acid (C14:0), isomyristic acid (C14:0), palmitic acid (C16:0), palmitoleic acid (C16:1), stearic acid (C18:0), oleic acid (C18:1), linoleic acid (C18:2), linolenic acid (C18:3), parinaric acid (C18:4), petroselinic acid (C18:1), or combinations thereof.
8 The composition of statement 6 or 7, wherein the C10-C18 fatty acid(s) comprise lauric acid (C12:0), myristic acid (C14:0), or a combination thereof.
9 The composition of any of statements 6 to 8, wherein the C10-C18 fatty acid(s) comprise palmitoleic acid (C16:1), oleic acid (C18:1), linoleic acid (C18:2), linolenic acid (C18:3), parinaric acid (C18:4), petroselinic acid (C18:1), or a combination thereof.
10 The composition of any of statements 6 to 8, wherein the composition is essentially free of unsaturated C10-C18 fatty acids.
11 The composition of any of statements 6 to 10, wherein the C10-C18 fatty acid(s) is/are present in the form of free acid, alkali metal salt, alkaline earth metal salt, ammonium salt or combination thereof, in particular wherein the fatty acid(s) is/are present in the form of free acid(s).
12 The composition of any of statements 6 to 11, wherein the one or more C10-C18 fatty acid(s) is/are present in an amount of from 0.010 to 10.0 percent-by-weight, in particular in an amount of 0.10 to 2.0 percent-by-weight, based on the weight of the composition.
13 The composition of any of statements 6 to 12, wherein the caffeine is present in an amount of from 0.010 to 10.0 percent-by-weight, in particular in an amount of 0.10 to 2.0 percent-by-weight, based on the weight of the composition.
14 The composition of any of the preceding statements, wherein the sandal pentanol is present in an amount of from 0.010 to 10.0 percent-by-weight, in particular in an amount of 0.10 to 2.0 percent-by-weight, based on the weight of the composition.
15 The composition of any of the preceding statements, comprising sandal pentanol in an amount of from 0.50 to 2.0 percent-by-weight, caffeine in an amount of from 0.50 to 2.0 percent-by-weight, and one or more C10-C18 fatty acid(s) in an amount of from 0.10 to 2.0 percent-by-weight, each based on the weight of the composition.
16 The composition of any of the preceding statements, comprising sandal pentanol in an amount of from 0.80 to 1.50 percent-by-weight, caffeine in an amount of from 0.80 to 1.50 percent-by-weight, and one or more C10-C18 fatty acid(s) in an amount of from 0.10 to 1.0 percent-by-weight, each based on the weight of the composition.
17 The composition of any of the preceding statements, comprising sandal pentanol and caffeine, in a weight ratio of sandal pentanol to caffeine from 1:2 to 2:1, in particular from 1:1.5 to 1.5:1, for example from 1:1.2 to 1.2:1.
18 The composition of any of the preceding statements, comprising sandal pentanol and one or more C10-C18 fatty acid(s), in a weight ratio of sandal pentanol to fatty acid(s) from 10:1 to 1:1, in particular from 8:1 to 1.5:1, for example from 5:1 to 2:1.
19 The composition of any of the preceding statements, further comprising a supportive component, in particular a supportive component selected from vasodilatators, blood circulation stimulators, antioxidants, vitamins, provitamins, and combinations thereof.
20 The composition of statement 19, wherein the supportive component is present in an amount of from 0.0010 to 5.0, in particular from 0.010 to 3.0 percent-by-weight, based on the weight of the composition.
21 The composition of statement 19 or 20, wherein the supportive component comprises one or more of tocopherols, tocotrienols, tocopherol esters, nicotinic acid, nicotinic amides, nicotinic esters, vasodilatory plant extracts, camphor, royal jelly, biotin, vitamin B3, vitamin B12, vitamin C, vitamin D2, vitamin D3, vitamin E, vitamin K1, vitamin K2, panthenol, omega-3 fatty acids, omega-6 fatty acids, folates, cannabidiol, amino acids or combinations thereof.
22 The composition of any of the preceding statements, further comprising an oil component selected from peppermint oil, rosemary essential oil, lavender essential oil, tea tree oil, ylang-ylang, thyme oil, cedarwood oil, clary sage essential oil, bergamot oil, jojoba oil, sunflower oil, hydrogenated ethylhexyl olivate, or a combination thereof.
23 The composition of statement 22, wherein the oil component is present in an amount of from 0.0010 to 1.0, in particular from 0.010 to 0.50 percent-by-weight, based on the weight of the composition.
24 The composition of any of the preceding statements, further comprising a mineral component selected from iron, zinc, or a combination thereof.
25 The composition of statement 24, wherein the mineral component is present in 0.0050-0.50 molar concentration.
26 The composition of any of the preceding statements, further comprising one or more cosmetically acceptable additives selected from perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents, or combinations thereof.
27 The composition of any of the preceding statements, further comprising a solvent.
28 The composition of statement 27, wherein the solvent is water-based, alcohol-based or water-alcohol-based.
29 The composition of statement 27 or 28, wherein the solvent comprises water and an alcohol selected from ethanol, propanol, glycerol, ethylene glycol, propylene glycol or a combination thereof.
30 The composition of any of statements 27 to 29, further comprising a solubilisation enhancer.
31 The composition of statement 30, wherein the solubilisation enhancer comprises one or more surfactants.
32 The composition of any of statements 27 to 31, having a pH within the range of from 3.0 to 8.0, in particular within the range of from 4.0 to 6.5, such as within the range of from 4.5 to 6.0.
33 The composition of any of statements 27 to 32, comprising 70.0 to 99.5 percent-by-weight, in particular 90.0 to 99.0 percent-by-weight, for example 92.0 to 98.0 percent-by-weight, based on the weight of the composition, water-alcohol-based solvent.
34 The composition of statement 33, comprising water in an amount of at least 10 percent-by-weight, based on the weight of the composition, and alcohol in an amount of at least 20 percent-by-weight, based on the weight of the composition.
35 The composition of statement 33 or 34, wherein the solvent comprises an alcohol selected from ethanol, n-propanol, isopropanol, glycerol, ethylene glycol, propylene glycol or a combination thereof.
36 The composition of any of statement 33 to 35, wherein the composition is a hair tonic.
37 The composition of any of statements 27 to 32, comprising a water-based solvent, and one or more surfactants in an amount of 5.0 to 25.0 percent-by-weight, based on the weight of the composition, wherein the surfactant(s) comprise(s) at least one anionic surfactant.
38 The composition of statement 37, wherein the composition is a shampoo.
39 The composition of any of statements 27 to 32, comprising an alcohol-based or water-alcohol-based solvent, and one or more surfactants in an amount of 5.0 to 45.0 percent-by-weight, based on the weight of the composition, wherein the surfactant(s) comprise(s) at least one anionic surfactant.
40 The composition of statement 39, wherein the solvent comprises an alcohol selected from ethanol, n-propanol, isopropanol, glycerol, ethylene glycol, propylene glycol or a combination thereof.
41 The composition of statement 39 or 40, wherein the composition is a shampoo.
42 The composition of any of statements 27 to 32, comprising 70.0 to 99.5 percent-by-weight, in particular 90.0 to 99.0 percent-by-weight, for example 92.0 to 98.0 percent-by-weight, based on the weight of the composition, of a blend comprising starch particles and an alcohol-based solvent.
43 The composition of statement 42, wherein the blend further comprises a propellant.
44 The composition of statement 42 or 43, wherein the blend further comprises inorganic particles.
45 The composition of any of statements 42 to 44, wherein the blend comprises, based on the weight of the blend, 5.0 to 12.0 percent-by-weight starch particles, 30.0 to 50.0 percent-by-weight alcohol-based solvent, 40.0 to 60.0 percent-by-weight propellant, and optionally 0.50 to 1.0 percent-by-weight inorganic particles.
46 The composition of any of statements 42 to 45, wherein the blend comprises, based on the weight of the blend, 7.0 to 10.0 percent-by-weight starch particles, 33.0 to 38.0 percent-by-weight alcohol-based solvent, 52.0 to 60.0 percent-by-weight propellant, and optionally 0.60 to 0.90 percent-by-weight inorganic particles.
47 The composition of any of statements 42 to 46, wherein the composition is a dry shampoo.
48 The composition of any of statements 27 to 32, wherein the composition is a ready-to-use scalp-and-hair treatment product selected from hair tonic, hair lotion, hair conditioner, hair cream, hair shampoo, dry shampoo, hair rinse, hair liquid, hair oil, hair pomade, hair foam, hair spray.
49 The composition of statement 48, wherein the product is a leave-on product.
50 The composition of any of the preceding statements, wherein the composition is essentially free of biotin.
51 The composition of any of the preceding statements, wherein the composition is essentially free of quaternary ammonium compounds.
52 The composition of any of the preceding statements, wherein the composition is essentially free of silicones.
53 The composition of any of the preceding statements, wherein the composition is essentially free of non-film-forming polymers.
54 The composition of statement 53, wherein the composition is essentially free of acrylates/Steareth-20 methacrylate copolymer, carbomer, polyacrylamide, hydroxyethylcellulose, Xanthan Gum.
55 A method for improving hair structure, wherein the method comprises applying sandal pentanol, or a composition of any of the preceding statements to at least part of the scalp region, or to at least part of a skin region comprising facial hair.
56 The method of statement 55, wherein improving hair structure comprises increasing the cross-section of the hair.
57 The method of statement 55 or 56, wherein improving hair structure comprises increasing the expression of keratin.
58 The method of statement 57, wherein improving hair structure comprises increasing the expression of keratin 85.
59 The method of any of statements 55 to 58, for improving the structure of hair affected by age-related hair thinning.
60 The method of any of statements 55 to 59, comprising applying sandal pentanol or the composition of any of statements 1 to 54 to at least part of the scalp region, or to at least part of a skin region comprising facial hair once or twice per day.
61 Use of the composition of any of statements 1 to 54 for improving hair structure.
62 Use of sandal pentanol (3-methyl-5-(2,2,3-trimethylcyclopent-3-en-1-yl) pentan-2-ol), for improving hair structure.
63 The use of statement 61 or 62, wherein improving hair structure comprises increasing the cross-section of the hair.
64 The use of any of statements 61 to 63, wherein improving hair structure comprises increasing the expression of keratin.
65 The use of statement 64, wherein improving hair structure comprises increasing the expression of keratin 85.
66 The use of any of statements 61 to 65, for improving the structure of hair affected by age-related hair thinning.
67 The use of any of statements 61 to 65, comprising applying sandal pentanol or the composition of any of statements 1 to 54 to at least part of the scalp region, or to at least part of a skin region comprising facial hair once or twice per day.

1. Composition comprising sandal pentanol (3-methyl-5-(2,2,3-trimethylcyclopent-3-en-1-yl) pentan-2-ol), for improving hair structure, wherein improving hair structure comprises increasing the cross-section of the hair.

2. The composition of claim 1, wherein improving hair structure comprises increasing the expression of keratin.

3. (canceled)

4. (canceled)

5. The composition of claim 1, further comprising caffeine, one or more saturated and/or unsaturated C10-C18 fatty acids, or a combination thereof.

6. The composition of claim 5, wherein the C10-C18 fatty acid(s) is/are selected from capric acid (C10:0), lauric acid (C12:0), myristic acid (C14:0), isomyristic acid (C14:0), palmitic acid (C16:0), palmitoleic acid (C16:1), stearic acid (C18:0), oleic acid (C18:1), linoleic acid (C18:2), linolenic acid (C18:3), parinaric acid (C18:4), petroselinic acid (C18:1), or combinations thereof.

7. (canceled)

8. (canceled)

9. The composition of claim 5, wherein the composition is essentially free of unsaturated C10-C18 fatty acids.

10. The composition of claim 5, wherein the C10-C18 fatty acid(s) is/are present in the form of free acid, alkali metal salt, alkaline earth metal salt, ammonium salt or combination thereof, in particular wherein the fatty acid(s) is/are present in the form of free acid(s).

11. The composition of claim 5, wherein the one or more C10-C18 fatty acid(s) is/are present in an amount of from 0.010 to 10.0 percent-by-weight, in particular in an amount of 0.10 to 2.0 percent-by-weight, based on the weight of the composition, the caffeine is present in an amount of from 0.010 to 10.0 percent-by-weight, in particular in an amount of 0.01 to 2.0 percent-by-weight, based on the weight of the composition, and the sandal pentanol is present in an amount of from 0.010 to 10.0 percent-by-weight, in particular in an amount of 0.10 to 2.0 percent-by-weight, based on the weight of the composition.

12. (canceled)

13. (canceled)

14. (canceled)

15. (canceled)

16. (canceled)

17. (canceled)

18. The composition of claim 1, further comprising a supportive component, in particular a supportive component selected from vasodilatators, blood circulation stimulators, antioxidants, vitamins, provitamins, and combinations thereof, in an amount of from 0.010 to 5.0, in particular from 0.010 to 3.0 percent-by-weight, based on the weight of the composition.

19. (canceled)

20. The composition of claim 18, wherein the supportive component comprises one or more of tocopherols, tocotrienols, tocopherol esters, nicotinic acid, nicotinic amides, nicotinic esters, vasodilatory plant extracts, camphor, royal jelly, biotin, vitamin B3, vitamin B12, vitamin C, vitamin D2, vitamin D3, vitamin E, vitamin K1, vitamin K2, panthenol, omega-3 fatty acids, omega-6 fatty acids, folates, cannabidiol, amino acids or combinations thereof.

21. The composition of claim 1, further comprising an oil component selected from peppermint oil, rosemary essential oil, lavender essential oil, tea tree oil, ylang-ylang, thyme oil, cedarwood oil, clary sage essential oil, bergamot oil, jojoba oil, sunflower oil, hydrogenated ethylhexyl olivate, or a combination thereof, in an amount of from 0.0010 to 1.0, in particular from 0.010 to 0.50 percent-by-weight, based on the weight of the composition.

22. (canceled)

23. The composition of claim 1, further comprising a mineral component selected from iron, zinc, or a combination thereof, present in 0.0050-0.50 molar concentration.

24. (canceled)

25. (canceled)

26. The composition of claim 1, further comprising a solvent wherein the solvent is water-based, alcohol-based or water-alcohol-based.

27. (canceled)

28. (canceled)

29. The composition of claim 26, further comprising a solubilisation enhancer, wherein the solubilisation comprises one or more surfactants.

30. (canceled)

31. The composition of claim 26, having a pH within the range of from 3.0 to 8.0, in particular within the range of from 4.0 to 6.5, such as within the range of from 4.5 to 6.0.

32. The composition of claim 26, comprising 70.0 to 99.5 percent-by-weight, in particular 90.0 to 99.0 percent-by-weight, for example 92.0 to 98.0 percent-by-weight, based on the weight of the composition, water-alcohol-based solvent, wherein the solvent comprises an alcohol selected from ethanol, n-propanol, isopropanol, glycerol, ethylene glycol, propylene glycol or a combination thereof.

33. (canceled)

34. (canceled)

35. (canceled)

36. The composition of claim 26, comprising a water-based solvent, and one or more surfactants in an amount of 5.0 to 25.0 percent-by-weight, based on the weight of the composition, wherein the surfactant(s) comprise(s) at least one anionic surfactant.

37. (canceled)

38. The composition of claim 26, comprising an alcohol-based or water-alcohol-based solvent, and one or more surfactants in an amount of 5.0 to 45.0 percent-by-weight, based on the weight of the composition, wherein the surfactant(s) comprise(s) at least one anionic surfactant.

39. (canceled)

40. (canceled)

41. The composition of claim 26, comprising 70.0 to 99.5 percent-by-weight, in particular 90.0 to 99.0 percent-by-weight, for example 92.0 to 98.0 percent-by-weight, based on the weight of the composition, of a blend comprising starch particles and an alcohol-based solvent.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. A method for improving hair structure, wherein improving hair structure comprises increasing the cross-section of the hair, wherein the method comprises applying sandal pentanol, or a composition of claim 5 to at least part of the scalp region, or to at least part of a skin region comprising facial hair.

55. The method of claim 54, wherein improving hair structure comprises increasing the expression of keratin.

56. (canceled)

57. (canceled)

58. (canceled)

59. (canceled)

60. (canceled)

61. (canceled)

62. (canceled)

63. (canceled)

64. (canceled)
